(19) United States
(12) Patent Application Publication
(10) Pub. No.: US 2015/0290418 A1
(43) Pub. Date: Oct. 15, 2015

(54) TREATMENT OF RESPIRATORY CONDITION USING TARGETED DELIVERY

(71) Applicants: David W. Kaczka, Iowa City, IA (US); Jarred R. Mondonedo, Boston, MA (US); Brett A. Simon, New York, NY (US)

(72) Inventors: David W. Kaczka, Iowa City, IA (US); Jarred R. Mondonedo, Boston, MA (US); Brett A. Simon, New York, NY (US)

(73) Assignee: Beth Israel Deaconess Medical Center, Inc., Boston, MA (US)

(21) Appl. No.: 14/511,590

(22) Filed: Oct. 10, 2014

Related U.S. Application Data
(60) Provisional application No. 61/889,966, filed on Oct. 11, 2013.

(51) Int. Cl. A61M 16/18; A61M 16/08; A61M 16/20; A61M 16/00

(52) U.S. Cl. CPC A61M 16/18; A61M 16/0003; A61M 16/0069; A61M 16/085; A61M 16/201; A61M 2016/003; A61M 2016/0027; A61M 2205/3334; A61M 2205/52; A61M 2230/432; A61M 2230/435; A61M 2230/437; A61M 2230/46; A61M 2209/084

(57) ABSTRACT

System and methods are provided for targeted delivery of agent for treatment of a respiratory condition. A respiratory characteristic is measured, and an agent is delivered based on the respiratory characteristic during a respiratory cycle. A computer controlled system is used to control mixing of an agent into the fluid delivery channel of a ventilator to treat the respiratory condition of a patient.

### CROSS REFERENCE TO RELATED APPLICATION

This application claims the priority of U.S. Provisional Application No. 61/889,966 filed Oct. 11, 2013, the entire contents of the above application being incorporated herein by reference.

### STATEMENT OF GOVERNMENT INTEREST

This invention was made with government support under Grant No. NIH HL089227. The government may have certain rights in this invention.

### BACKGROUND

Status asthmaticus (SA) is a severe, refractory form of asthma that can result in rapid respiratory deterioration and death. Volatile anesthetics are often used as treatment for SA, especially in pediatric patients. However, these agents can induce significant adverse side effects, notably sedation, hypotension, and arrhythmias, especially during prolonged use. Because of these side effect, there is a limit on the dose that can be delivered to a patient. Additionally, traditional treatment systems require either the use of an anesthetic machine, with limited modern ventilator capabilities, or the ad hoc rigging of an administration system to an Intensive Care Unit (ICU) ventilator, neither of which is sufficient to achieve safe and effective delivery while avoiding adverse side effects.

### SUMMARY OF THE INVENTION

The present invention relates to the treatment of respiratory conditions. Preferred embodiments of the invention include systems and methods for delivering one or more medications into the respiratory system of human or animal subjects. A control system is used to actuate a valve assembly in which a specified treatment dosage is correlated with the respiratory cycle of a patient. The system can include an apparatus that is used to measure a respiratory characteristic. An agent or medication is delivered based on the respiratory characteristic during a respiratory cycle. A preferred embodiment includes a data processing system such as a portable computer to control system operation and record measured data.

Measuring a respiratory characteristic can include measuring one or more of an airway pressure, an airway flow, a percent of anesthetic agent present, a carbon dioxide partial pressure, an oxygen partial pressure, and the like. Measuring a respiratory characteristic may also include determining a breath phase. The breath phase may be determined based on comparing an airway flow to various thresholds. Based on the comparison and the previous breath phase, the breath phase is determined to be either an inspiration or expiration.

The measuring of a respiratory characteristic can be triggered by a ventilator cycle, such as when the subject is breathing with the aid of a ventilator. The agent or medication delivered to the subject may include an anesthetic. The present invention can be used generally for the treatment of asthma, and in particular for the treatment of status asthmaticus. Additionally, lung resistance, lung elastance, and anatomic dead space can be determined based on one or more respiratory characteristic(s). The respiratory characteristic(s) can be re-measured after the delivery of the agent to determine the effect of the treatment.

### BRIEF DESCRIPTION OF THE DRAWINGS

FIG. 1 is a schematic illustrating an exemplary system for treatment of a respiratory condition using targeted delivery, according to an example embodiment.

FIG. 2 is a diagram illustrating an exemplary control circuit included in a system for treatment of respiratory condition using targeted delivery, according to an example embodiment.

FIG. 3 is a diagram illustrating exemplary system comprising modules included in a system for treatment of respiratory condition using targeted delivery, according to an example embodiment.

FIG. 4 is a flowchart illustrating an exemplary method for treatment of respiratory condition using targeted delivery, according to an example embodiment.

FIG. 5 is a flowchart illustrating an exemplary method for breath detection includes in a method for treatment of respiratory condition using targeted delivery, according to an example embodiment.

FIG. 6 are exemplary graphs illustrating techniques for estimating total delivered and exhaled volume of anesthetic, according to an example embodiment.

FIG. 7 are exemplary graphs illustrating various lung measurements for targeted isoflurane delivery measured during various phases of treatment, according to an example embodiment.

FIG. 8 is an exemplary graph illustrating results of various delivery modes of sevoflurane and isoflurane in comparable levels of bronchodilation, according to an example embodiment.

FIG. 9 are exemplary graphs illustrating isoflurane traces during various delivery modes in relation to the delivered tidal volume for two breaths, according to an example embodiment.

FIG. 10 is a diagram illustrating an exemplary computing system for treatment of respiratory condition using targeted delivery, according to an example embodiment.

### DETAILED DESCRIPTION OF PREFERRED EMBODIMENTS

Systems and methods are provided for treatment of respiratory conditions using targeted delivery of agents in a patient. In a preferred embodiment, targeted delivery of inhalational anesthetics to the anatomic dead space of the patient's airways maximizes their bronchodilator effects while minimizing systemic absorption and side effects. One preferred embodiment is an anesthetic-ventilator system comprising a computer-actuated ventilator-valve system for the targeted delivery of inhalation anesthetics at selective time intervals during inspiration. One or more respiratory characteristics or parameters are measured, and the agent is delivered based on the measured characteristics or parameters. This optimized delivery regime can provide improved treatment paradigms for this life-threatening condition, while increasing the understanding of the mechanism of action for inhalation anesthetic agents.

Targeted delivery of the anesthetic agent is achieved by regulating the serial composition of the inspired gas. The ventilator-valve circuit may be designed to divert at least the last portion of the inspired volume to the anesthetic vaporizer. The precise timing and ratio between bypass ventilation and anesthetic delivery may be performed in real-time using the actuating software that developed for this application. In an example embodiment, the anesthetic-ventilator system is self-contained on a small portable cart that requires few external connections, making it ideal for use in an environment like an intensive care unit (ICU). In alternative embodiments, the anesthetic-ventilator system may not be self-contained, and may be installed at a particular location so that it is easily accessible by healthcare professionals for a particular patient. For example, the anesthetic-ventilator may be non-portably installed near a patient bed.

The ventilator requires, at least, a power source, and air and oxygen sources, for example. Prior to treatment, the anesthetic agent, in liquid form, is loaded into the vaporizer, and the target delivery volume of the anesthetic can be manually adjusted at any time during use via the user-friendly control software. In addition to pressure, flow, and volume, the software can be programmed to display other parameters for real-time patient monitoring. These parameters can include, but are not limited to, carbon dioxide partial pressure (pCO₂), oxygen partial pressure (pO₂), tidal volume (V_{T}), dead space volume (V_{D}), respiratory rate (RR), and minute volume (MV).

In another example embodiment, the valve assembly system is mounted on an table that can be easily transported and connected to any ICU ventilator. The system can also be configured in a hand-carried housing for easier transport.

In alternative embodiments, the system described herein can be used with other inhaled particles, aerosols, or medications to treat other conditions. The inhaled particles, aerosols, and medications can include, but are not limited to, beta agonists, steroids, glucocorticoids, anticholinergics, and leukotriene inhibitors. The system can be programmed to accommodate these other substances. Depending on the substance used, the system may not require all the components described above to enable controlled delivery of the agent or substance.

In a preferred embodiment, selective delivery to a target volume is achieved by actuating two solenoid valves, which control the composition of the inspired gas, at specified intervals during inspiration. Precise timing between bypass ventilation and anesthetic delivery can be performed in real-time using actuating software. The actuating software may be developed using any suitable programming language or platform, such as, LabVIEW, for example.

Various data can be measured as part of the systems and methods described herein. Airway pressure (P) and flow (\dot{V}) can be measured using any suitable equipment, such as, a standard pneumotachograph. One such pneumotachograph is the RSS 100-HR made by Hans Rudolph, Inc., Kansas City, Mo. Anesthetic agent (AA), oxygen (O₂), and carbon dioxide (CO₂) gas concentrations can be simultaneously monitored using any suitable equipment, such as, a side-stream gas analyzer. One such gas analyzer is a Datex-Ohmeda made by GE Healthcare, Fairfield, Conn.

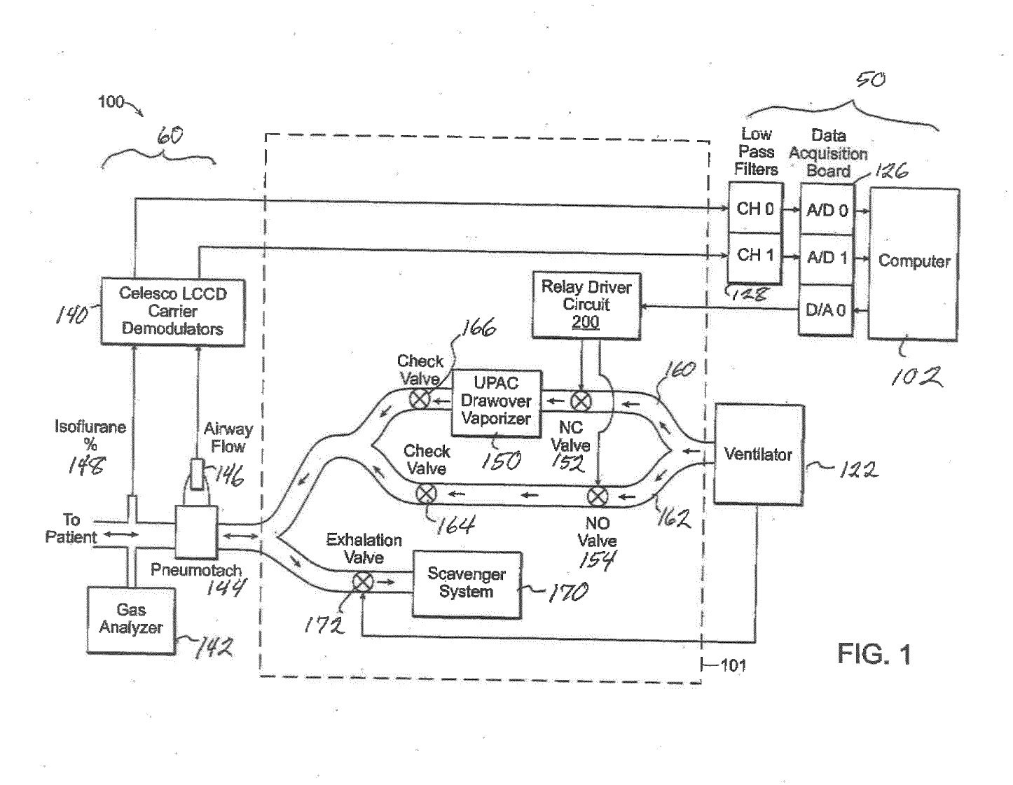

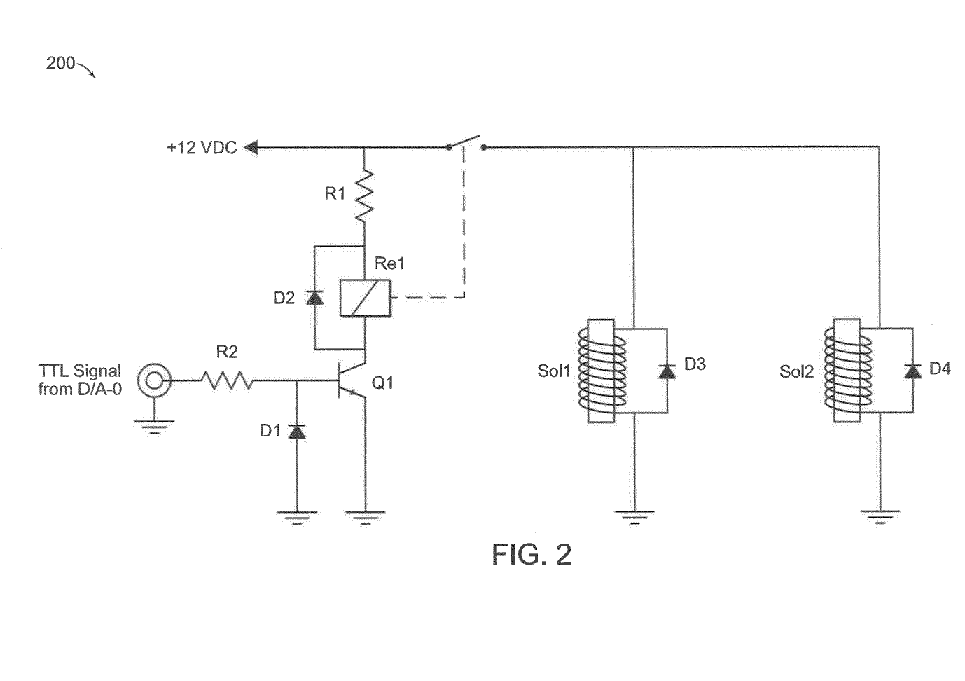

In a preferred embodiment, the system includes a ventilator valve circuit. The ventilator in the anesthetic ventilator system is not limited to any particular ventilator, and can be used with any available ventilator. In an example embodiment, the Model 754 Eagle made by Impact Instrumentation Inc., West Caldwell, N.J., may be used. In an example embodiment, the inspiratory limb is divided into at least two channels such that the carrier gas either passes uninhibited, or is pushed over the vaporizer (as shown in FIG. 1, discussed below). The flow can be moderated by two solenoid valves (as shown in FIG. 1) driven by a relay circuit (shown in FIG. 2, discussed below). The vaporizer may be a UPAC Drawover Vaporizer, for example. The reduced size and rapid response time of the UPAC Drawover Vaporizer benefits the method for targeted anesthetic delivery in ICU. The UPAC Drawover Vaporizer is specifically designed for delivering volatile anesthetics. In alternative embodiments, substances other than volatile anesthetics may be delivered using a nebulizing chamber instead of a vaporizer.

The system may also include actuating software installed on the computer 102 shown in FIG. 1, discussed below. In a preferred embodiment, a customized software interface provides user-control of targeted or continuous delivery of inhalation anesthetics to a known volume by specifying a delivery window during inspiration.

The tidal volume can be calculated in real-time by trapezoidal integration of \dot{V} using the following formula, for example:

$${V}_{t} ( i ) = \frac{1}{2} \{ [ t ( i ) - t ( i - 1 ) ] \cdot [ \dot{V} ( i ) - \dot{V} ( i - 1 ) ] \} + {V}_{t} ( i - 1 )$$

Slow drifts and transient fluctuations can introduce considerable errors in the estimation of Vₜ. To overcome these errors, some embodiments use breath-by-breath analysis. Individual breaths are detected by identifying inspiratory/expiratory regions characteristic of mechanical ventilators. The logic network provides additional stability for breath detection.

Measurements can be obtained using a mechanical lung provided by Ingmar Medical, Pittsburgh, Pa., simulating the ‘lung parenchyma,’ in series with a known volume of corrugated tubing simulating an interchangeable ‘dead space’ compartment. The mechanical lung may consist of 180 cc of corrugated tubing dead space in series with two spring-loaded bellows representing the alveolar compartment. These measurements indicate that the anesthetic ventilator system is operative to treat bronchodilator effects while minimizing systemic absorption and side effects. The anesthetic ventilator system can be used to selectively deliver agent to the mechanical lung for typical canine and human ventilation rates and tidal volumes.

TABLE 1
Ventilation Parameters

| Group | Tidal Volume (mL) | Ventilation Rate (min⁻¹) |
|---|---|---|
| Canine Test Lung | 250 | 10, 20, 30 |
| | 375 | 10, 20, 30 |
| | 500 | 10, 20, 30 |
| Human Test Lung | 400 | 08, 10, 12 |
| | 600 | 08, 10, 12 |
| | 800 | 08, 10, 12 |

Severe sustained bronchospasm may be established using continuous i.v. infusion of methacholine. Using the anesthetic ventilator system, a user has the ability to control the timing, peak concentration, and duration of inhaled isoflurane delivery for various ranges of conventional ventilator rates and tidal volumes typical of ICU patients. In one embodiment, the solenoid valves (shown in FIG. 1) can be programmed to deliver isoflurane during the last 180 cc of the delivered inspired volume. Furthermore, none of the inhaled isoflurane delivered during the last portion of the inspiratory period reaches the alveolar compartment to reduce the risk of side effects and systemic absorption.

FIG. 1 is a schematic illustration of an exemplary system 100 for treatment of a respiratory condition using targeted delivery, according to an example embodiment. In system 100, arrows within the ventilator circuit denote direction of flow. The system 100 includes several components including a data collection and control component 50, a fluid delivery and detection component 60, a ventilator 122, and medication control component 101. The fluid controller 101 includes a bypass flow valve, such as an NO Sol. valve, 154 which is a normally-open solenoid valve and first valve, such as an NC Sol. valve, 152 which is a normally-closed solenoid valve. The data collection component 50 can have two or more channels having low pass filters 128 which output filtered data to a data acquisition circuit board 126 having A/D circuits (analog-to-digital converters). Board 126 can also have D/A (digital-to-analog converter) circuitry to deliver control signals from computer 102 to a driver circuit 200 that is operative to control valves 152, 154. Check valves 164, 166 are included in the fluid control circuit to prevent back flow into the inspiratory limb as the gas travels to the Scavenger system 170 during expiration. The ventilator 122 can be connected to a valve 172 to control fluid delivery to system 170. The region enclosed in the dotted line is packaged in a separate housing system 101. The fluid control system 101 refers to the components of system 100 that can be packaged in a discrete housing that fits on an ICU cart. Housing system 101 can be hand carried with a handle attached to housing 101. Packaged system 101 may be considered a black box that has appropriate connections for the components outside the black box. As such, components within packaged system 101 can be provided separately from the other components in system 100. The other components, such as, ventilator 122, gas analyzer 142, pneumotach 144, and computer 102, may be available in an ICU room, while the packaged system 101 components, such as, relay driver circuit 200, a medication delivery device such as a drawover vaporizer 150, check valves, solenoid valves, exhalation valve, and Scavenger system, may be placed on an ICU cart that is wheeled into the ICU room when needed. The gas analyzer 142 measures gases present in the flow such as oxygen, carbon dioxide and optionally the anesthetic agent(s) present. The analyzer 142 output data can be delivered to computer 102 and/or stored and displayed on a separate system. The pneumotach 144 can comprise a differential pressure transducer 146 that generates an output signal for demodulator system 140 which is also operative to measure the percentage of an anesthetic in flow 148. The system 100 includes the computer 102 that has actuating software installed thereon to control the delivery of medication as described herein. System 100 also includes the relay driver circuit 200, illustrated in FIG. 2, that can be included in packaged system 101 that enables switching between two flow channels 160, 162.

FIG. 2 is a diagram illustrating an exemplary circuit 200 included in a system for treatment of a respiratory condition using targeted delivery agents, according to an example embodiment. The input to circuit 200 is a TTL signal from the digital-analog converter on board 126 in system 50 from FIG. 1. Circuit 200 includes resistors, diodes, a transistor, power supply, a switch and solenoids in electrical communication as illustrated in FIG. 2. When the resistor (R2) is high, the transistor Q1 is saturated, which in turn, closes the switch.

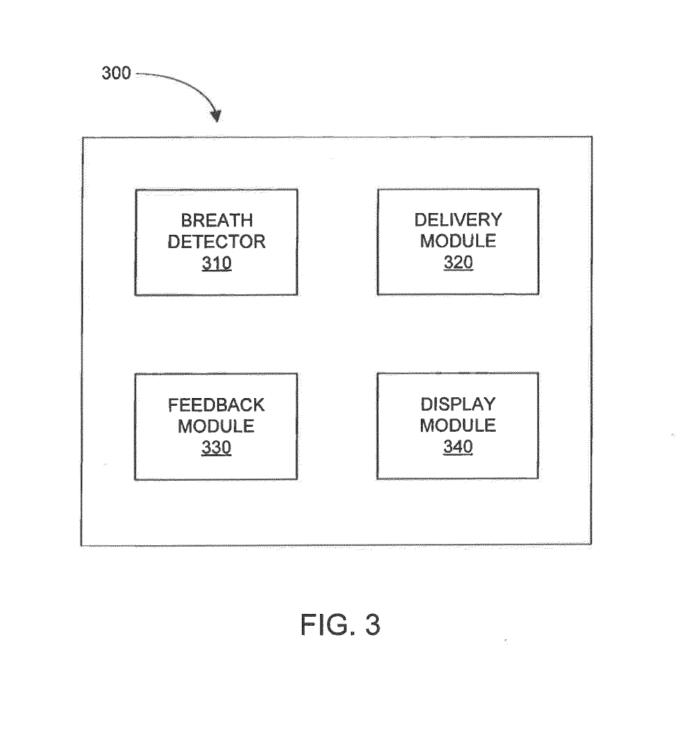

FIG. 3 is a schematic illustrating exemplary system 300 comprising modules included in a treatment of a respiratory condition using targeted delivery, according to an example embodiment. System 300 includes a breath detector 310, a delivery module 320, a feedback module 330, and a display module 340. System 300 may be implemented on the computer 102 that is configured to perform functions as described herein in connection with FIGS. 1 and 2 using methods described in greater detail below.

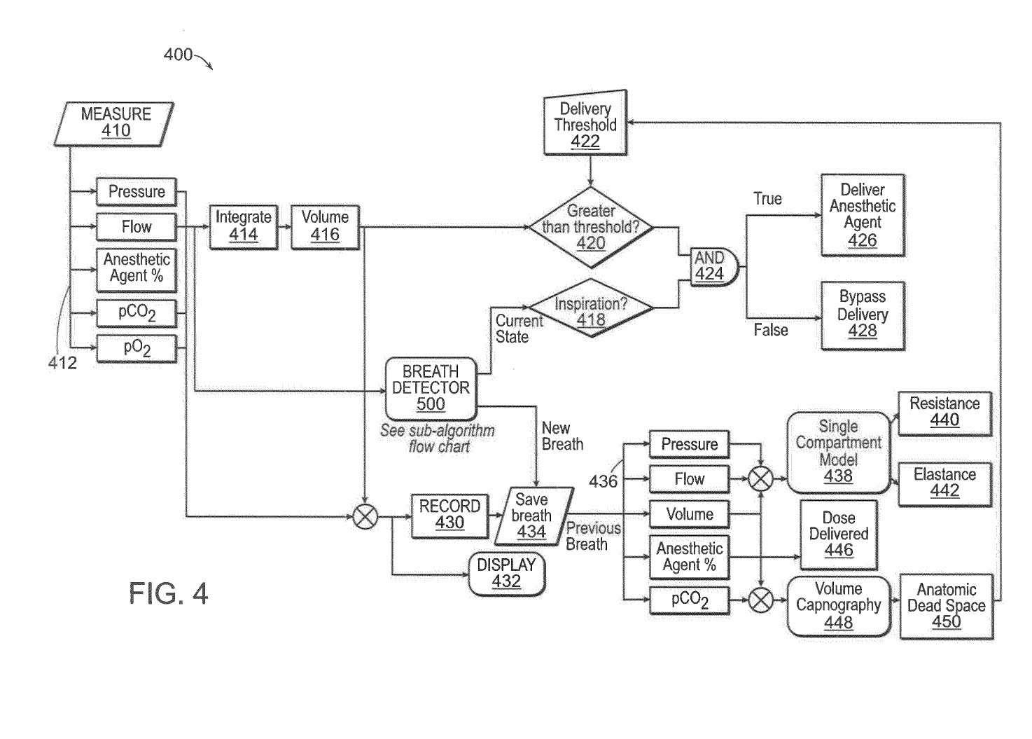

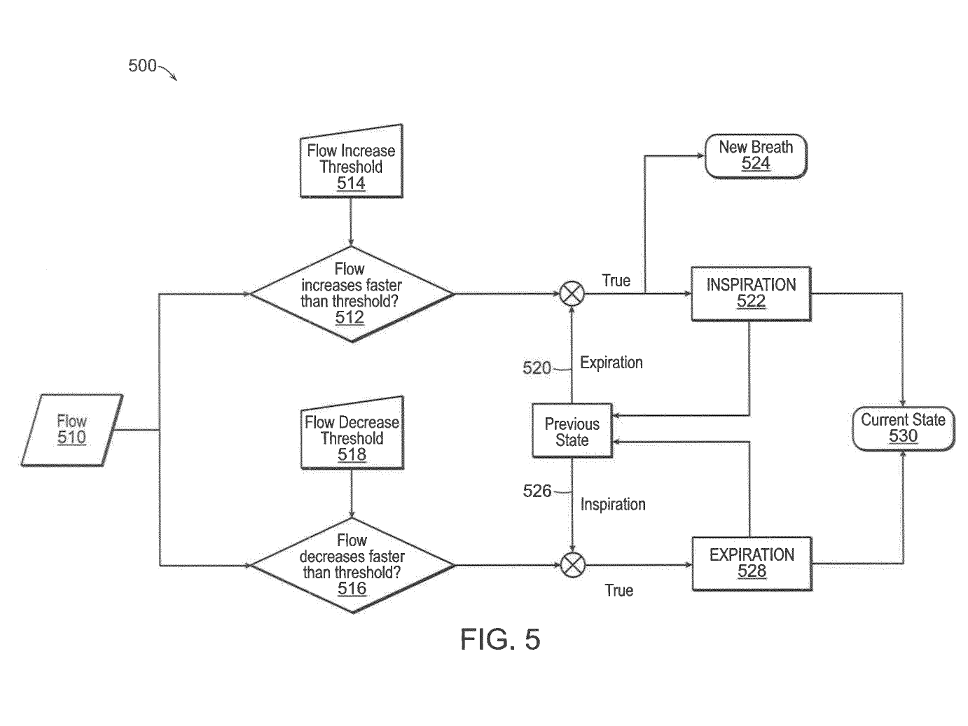

FIG. 4 is a flowchart illustrating an exemplary method 400 for treatment of a respiratory condition using targeted delivery, according to an example embodiment. FIG. 5 is a flowchart illustrating an exemplary method 500 for breath detection, according to an example embodiment. Methods 400 and 500 may be performed by the modules of system 300.

Referring to FIG. 4, method 400 starts with measuring (410) various parameters (412), such as, pressure, flow, percent of anesthetic agent present in the lungs, carbon dioxide partial pressure, and oxygen partial pressure. The pressure may be transpulmonary airway pressure. The pressure and flow and can be measured using a pneumotachograph as discussed above. The percent of anesthetic agent, carbon dioxide partial pressure, and oxygen partial pressure can be measured using a gas analyzer as discussed above. The measured flow is integrated (414) to derive the tidal volume waveform (416). The measured flow is input to the breath detector routine 500 shown in FIG. 5.

Method 500, the breath detector routine, determines whether the measured flow (510) is increasing faster than a flow increase threshold (514). If the flow increases faster than the threshold (512) and the previous breath was an expiration (520), then the current breath is determined to be an inspiration (522). The current breath is saved as a new breath (524). Method 500 also determines if the flow (510) is decreasing faster than a flow decrease threshold (518). If the flow decreases faster than the threshold (516) and the previous breath (526) was an inspiration, then the current breath is determined to be an expiration (528). The current state (530) is the output of the breath detector routine. The current state (530) is determine to be an inspiration or an expiration as described above. The thresholds (514 and 518) may be a predefined number that can depend on the various lung and breath characteristics.

After the breath detector routine 500 outputs the current state (530), method 400 continues by determining if the current state is an inspiration (418). Method 400 also determines whether the volume (416) is greater than a delivery threshold (422). If the current state is an inspiration (418) and the volume is greater than a delivery threshold (420), then an anesthetic agent is delivered (426) to the patient. If both these conditions (418 and 420) are not met, then the delivery of the agent is bypassed (428). Thus, an anesthetic agent is only delivered on an inspiration portion of the breath and after the volume is greater than a threshold. The threshold (422) is a predefined number so that everything within the airways' dead space remains there. For example, the volume of a general breath is 500-600 cubic centimeters, and the threshold may be set such that delivery of the anesthetic agent occurs when the breath volume is 120 cubic centimeters.

In addition to determining when to deliver the anesthetic agent, method 400 also records (430) the pressure, percent of anesthetic agent, carbon dioxide partial pressure and oxygen partial pressure, and displays (432) these parameters for a user to monitor. These parameters are further saved (434) along with the new breath. The saved breath (434) is used to determine the current parameters (436)—pressure, flow, volume, percent of anesthetic agent, and carbon dioxide partial pressure. The pressure, flow, and volume are used in a single compartment model (438) to represent lung resistance (440) and lung elastance (442). The percent of anesthetic agent is saved as the dose delivered (446). The carbon dioxide partial pressure and volume are used to determine the anatomic dead space (450) using volume capnography (448). The anatomic dead space (450), determined from the current parameters, is used as feedback in determining the delivery threshold (422).

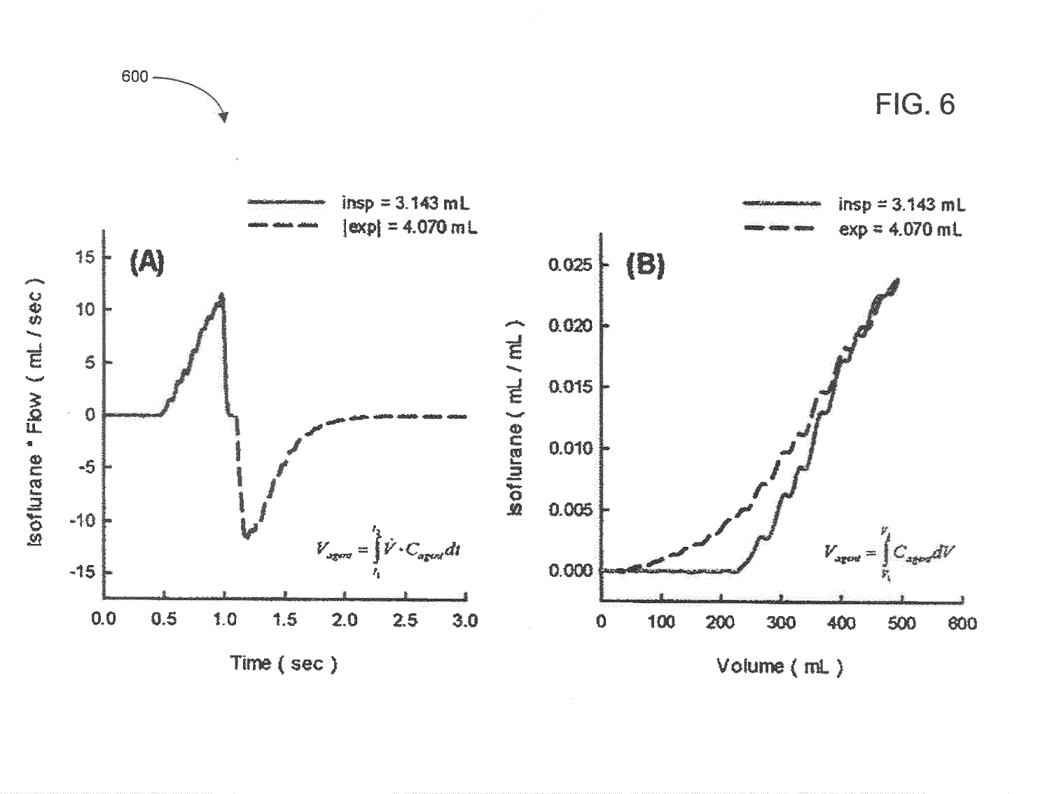

FIG. 6 are exemplary graphs 600 illustrating techniques for estimating total delivered and exhaled volume of anesthetic, according to an example embodiment. In an example embodiment, the delivered medication is an anesthetic, such as isoflurane and/or sevoflurane. In other embodiments, the delivered medication can be any other suitable medication that needs to be delivered to a patient for the treatment of a respiratory condition. In yet other embodiments, the delivered medication can be any medication suitable to treat other illnesses in a patient. Where the delivered medication is an anesthetic, total agent delivered and exhaled volume of medication can be determined using at least two techniques.

One of the techniques, illustrated in graph A of FIG. 6, is to integrate flow x isoflurane concentration over time:

$${V}_{agent} = {\int }_{t 1}^{t 2} \dot{V} \cdot {C}_{agent} d t$$

Another technique, illustrated in graph B of FIG. 6, is to integrate isoflurane concentration over volume:

$${V}_{agent} = {\int }_{V 1}^{V 2} {C}_{agent} d V$$

C_{agent} is the concentration of anesthetic agent in mL per mL of total gas volume. V_{agent} is the total volume of delivered anesthetic agent in mL. \dot{V} is flow in mL per second.

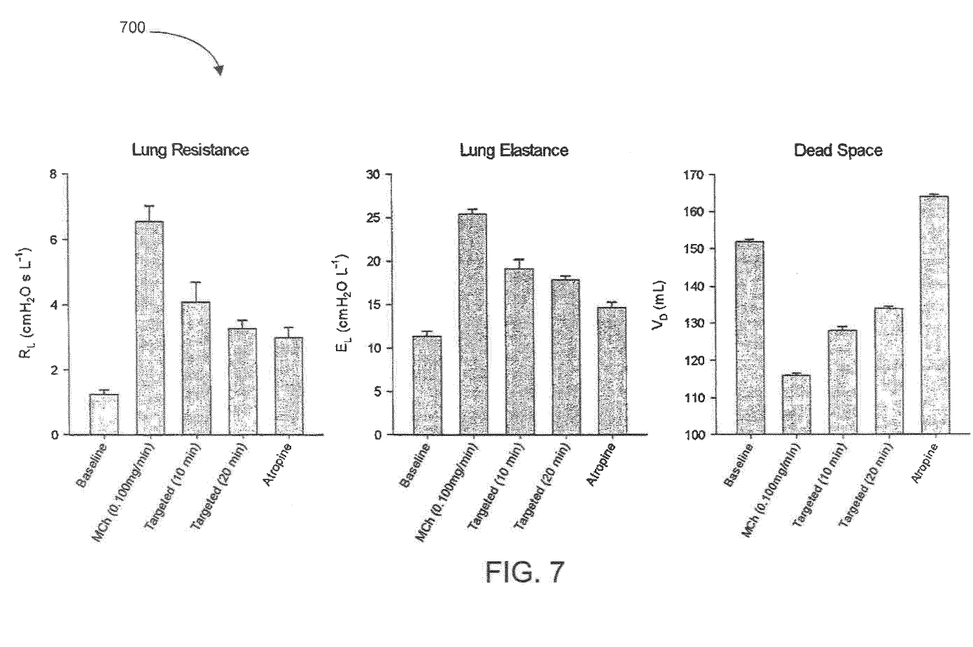

FIG. 7 are exemplary graphs 700 illustrating various lung measurements for targeted isoflurane delivery measured during various phases of treatment, according to an example embodiment. As shown in graphs 700, various lung measurements, including lung resistance (R_{L}), lung elastance (E_{L}), and dead space (V_{D}), are measured as described above. As shown in graphs 700, there is a decreasing trend in lung resistance and lung elastance following targeted therapy with isoflurane, while the increasing estimation of dead space denoting further evidence of bronchodilation in the airways. The estimates of lung resistance and lung elastance may be obtained via multiple linear regression on measured flow and transplumonary pressure waveforms. Anatomic dead space may be estimated using breath-by-breath volume capnography. Capnography is the monitoring of the concentration or partial pressure of carbon dioxide in the respiratory gases. These measurements may be acquired at least under baseline conditions, during bronchoconstriction, after 10-20 minutes of volatile anesthetic treatment, and after maximal bronochodilation with i.v. atrophine. Airway constriction results in increased airway resistance and stiffening of the lung parenchyma, which is often measured with lung resistance and lung elastance. Measuring these parameters help in determining the effectiveness of the targeted delivery treatment.

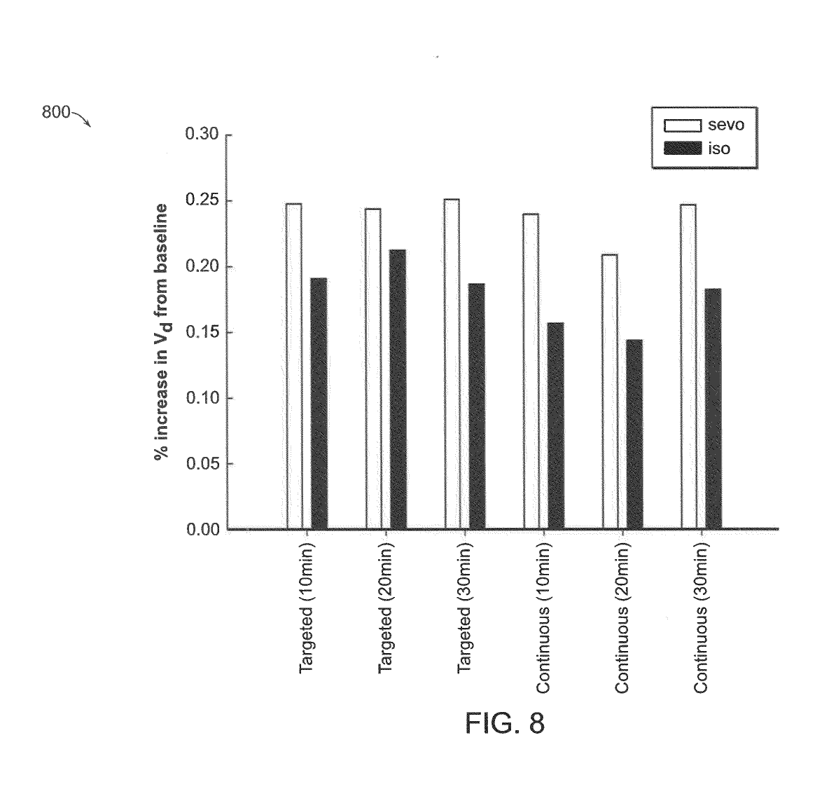

FIG. 8 is an exemplary graph 800 illustrating results of various delivery modes of sevoflurane and isoflurane in comparable levels of bronchodilation, according to an example embodiment. The various delivery modes are at least targeted mode and continuous mode. As shown in graphs 800, targeted delivery mode of either sevoflurane or isoflurane results in slightly higher bronchodilation than the continuous delivery mode.

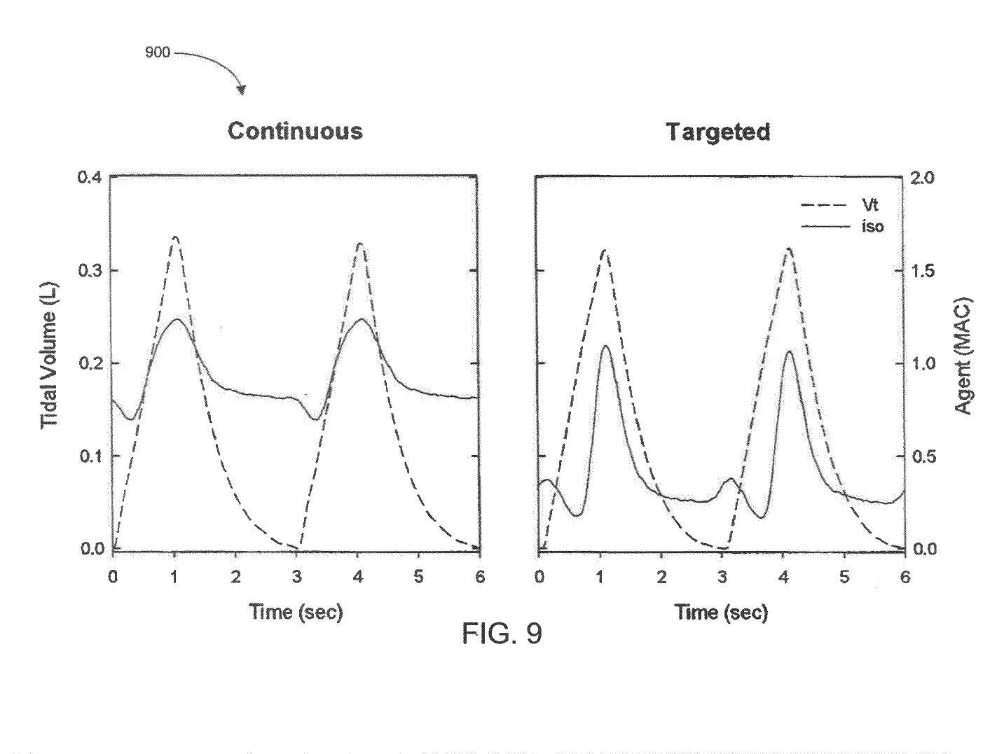

FIG. 9 are exemplary graphs 900 illustrating isoflurane traces during various delivery modes in relation to the delivered tidal volume for two breaths, according to an example embodiment. As shown in graphs 900, the solid line represents the amount of isoflurane in the lungs and the dotted line represents the tidal volume (Vt) over time. The levels of isoflurane agent is lower for most of the time in the targeted delivery method than the continuous delivery method. However, the targeted delivery method has comparable peak level of isoflurane to the continuous delivery method.

Using the systems and methods described here, volatile anesthetics can be selectively delivered for a specified interval during inspiration. Anesthetic agent delivery can be restricted to the anatomic dead space compartment. Residual anesthetic within the ventilator circuit may be eliminated on expiration, minimizing the potential for rebreathing. Additionally, the concentration gradient of delivered anesthetic decreases with distance, such that the upper airways receive larger anesthetic doses when this is advantageous to treat a particular condition. Measurements show that targeted and continuous delivery of volatile anesthetics result in comparable levels of bronchodilation. Targeted delivery of volatile anesthetics can be the treatment paradigm for status asthmaticus while increasing patient safety.

In other embodiments, measurements can be obtained using a canine model of severe bronchospasm and independent lung ventilation with a double-lumen endobronchial tube. In an example embodiment, measurements can be used to determine whether the bronchodilating effects of inhaled anesthetics is via direct diffusion-mediated effects on the airway smooth muscle, or it depends on uptake and distribution via the systemic circulation. By using an Enhanced Ventilator Waveform (EVW), the mechanical input impedance spectra of the respiratory system during ventilation can be continuously monitored, and the resistance and elastance spectra can be measured. Additionally, the heterogeneity of airway constriction can also be measured. In some embodiments, a functional CT imaging can be used to: 1) measure the effects of such interventions on luminal diameters throughout the airway tree; 2) track the actual distribution of the gas injection scheme with radiodense Xenon (Xe); and 3) measure mechanical interdependent effects through sophisticated image registration analysis. These measurements may help to determine the in vivo mechanisms of action of inhaled anesthetic agent treatment and provide a rapidly translatable treatment paradigm for respiratory disorders.

All inhaled agents appear to have similar bronchodilating properties, although isoflurane is preferred in some embodiments because of real or perceived side effect profiles. While treatment generally results in improvement within 12 hours, isoflurane has been used successfully for days in refractory cases. Because these drugs are systemically absorbed, they have predictable side effects including cardiac depression and arrhythmias, vasodilation, cerebral vasodilation, trigger for malignant hyperthermia, potential nephrotoxicity, and hepatotoxicity, all of which can limit dose or require treatment. Withdrawal effects have been noted after prolonged administration for ICU sedation. Moreover, anesthetics and their sedative effects make their use in unintubated patients risky.

Inhaled anesthetics can cause direct relaxation of airway smooth muscle in vitro through various mechanisms that reduce intracellular calcium availability, including inhibition of protein kinase C, calcium release from sarcoplasmic reticulum, and voltage-dependent calcium channels. In vivo, airway tone is regulated by the balance of parasympathetic-mediated constriction, sympathetic dilation, and non-adrenergic non-cholinergic neural input. Inhaled anesthetics may cause bronchodilation in vivo in part through reduction in vagal tone and reflexes, and may also affect circulating catecholamines or airway beta (β) receptor sensitivity. For direct action, the anesthetic gas need to get to the constricted airway, by definition on a poorly ventilated pathway, and then diffuse into the airway wall to reach the smooth muscle layer. Since inhaled anesthetics are relatively insoluble and have high molecular weights, their uptake into the conducting airways is less than for soluble gases. Based on these characteristics, there may be at least two viable routes for delivery: local diffusion or bronchial circulation. Both routes may be important, as β-agonists, the standard therapy for bronchospasm, work whether they are administered by aerosol (primarily to larger airways) or systemically. The bronchial circulation can in some embodiments have a role in facilitating drug delivery to regions distal to the original airway site of deposition or to non-ventilated lung regions as well as clearing agents that cause constriction, and thus can be important in delivering bronchodilating anesthetic to severely constricted or obstructed airways. The system and measurements described herein show whether sufficient direct uptake of agent through the airways to attain an effect is necessary, and further whether systemic absorption is necessary for a full therapeutic effect (i.e., delivery to poorly ventilated regions and central reflex depression vs. direct smooth muscle relaxation).

Airway constriction results in increased airway resistance and stiffening of the lung parenchyma, traditionally measured using single parameter resistance (R) and elastance (E) that imply a lung model consisting of a single tube leading to an elastic balloon. In an example embodiment, the lung is understood to be a distributed branching network of airways that constrict heterogeneously. Using lung impedance (Z), the relationship between instantaneous pressure and flow as a function of oscillation frequency, in conjunction with complex distributed models of the airway tree, allows for estimation of both the degree and the heterogeneity of airways constriction as well as changes in lung stiffness. In one embodiment, Z is measured using an Enhanced Ventilator Waveform (EVW) in which small flow oscillations at optimally chosen frequencies are superimposed on larger amplitude tidal volume changes such that Z can be measured continuously with every breath during conventional ventilation. Another embodiment consist of a computer-controlled ventilator that employs a high fidelity flow-controlling valve to implement EVW-type flow patterns, and it may be used to control the composition of inspired gas.

Lung imaging extends beyond visualizing anatomy and allows quantification of regional physiologic processes and dynamic lung function, including ventilation, perfusion, and tissue mechanics. In addition, airways may be analyzed in 3-D and the length, diameter, and wall thickness of pathways serving different lung regions can be semi-automatically extracted and compared. Image registration, in which a lung image at one volume is “warped” to a second, such as from expiration to inspiration, allows the calculation of local parenchymal expansion and thus tissue stiffness. One embodiment approach links oscillatory impedance and modeling heterogeneity, CT-based regional lung mechanics, and optimal ventilator management strategies in acute lung injury, making the extension of these approaches to status asthmaticus beneficial.

Interdependence effects, in which parenchymal tethering of airways and expansion of the surrounding lung helps maintain a positive transmural pressure across the airway wall and keep airways open, can create a paradoxical feedback mechanism promoting airway closure in large clusters. This phenomenon can be demonstrated using an imaged-based modeling approach, in which uniform smooth muscle activation with a small imposed heterogeneity resulted in catastrophic closure of large volumes of lung, with airways closure potentially occurring at the level of the small bronchioles. This same model helps predict that recruitment of the lung also proceeds in cascades of large clusters and further, that delivery of bronchodilators to already well-ventilated regions could exacerbate ventilation heterogeneity by reducing the interdependent expansion of constricted areas.

Different portions of the inspired gas volume can have different gas concentrations is familiar to anesthesiologists using traditional “Mapleson” style semi-open breathing circuits. Similar passive systems have been employed to control PₐC0₂ in exercise testing. Active systems, in which flow rate and breath timing are sensed and then aerosols or nitric oxide injected at a specific point in the breathing cycle, can be successfully used to improve delivery to specific lung zones or maintain drug concentration during changing flows. Challenges to selective delivery of anesthetic gas to conducting airways include gas mixing at the interface and parallel heterogeneity of ventilation distribution, which could result in long time constant regions receiving agent intended for conducting airways.

In some embodiments, severe sustained bronchospasm is induced in anesthetized, intubated canines and characterize the model in terms of temporal stability, oscillatory impedance, airways diameters, and tissue stiffness. The dose-response curve of various volatile anesthetic agents (such as, halothane, isoflurane, enflurane, sevoflurane) delivered throughout the entire inspiratory period, or selectively to the conducting airways is compared. Direct diffusion-mediated dilation of airway smooth muscle is distinguished from circulatory delivery by altering the timing of anesthetic delivery as detailed below. First, a severe bronchospasm is induced using intravenous histamine, and the bronchodilator dose response for each of the inhaled agents is compared while delivered throughout inspiration or restricted to the anatomic deadspace. Using anesthetic-ventilator delivery system, bronchoconstriction with the EVW and continuous Z measurement is assessed. Simultaneously, respiratory-gated CT imaging (GE Lightspeed VCT 64-detector scanner) is used at multiple time-points to examine the airway generation-diameter relationships, and registration analysis to determine the topographic distribution of local parenchymal compliances is used. To further differentiate direct versus systemic delivery, intravenous agonist delivery can be used to create bilateral bronchospasm and then independent lung ventilation to deliver anesthetic agent to only one lung, such that the contralateral lung receives agent only through the circulation. The end-tidal agent concentration is measured and Z is monitored independently for each lung using a double lumen endotracheal tube. To determine the relative bronchodilatory contributions of the anesthetics via the bronchial circulation versus vagal outflow, measurements can be obtained before and after intravenous atropine.

The resolution of severe bronchoconstriction in response to inhalational anesthetics agent in terms of oscillatory impedance, CT measurement of airways lengths and diameters, as well as CT registration of regional tissue mechanics are monitored. From these measurements, it can be determined whether airway “recruitment” or relaxation occurs in cascades along pathways, as functions of airways size, or in other spatial-temporal patterns. The effects of anesthetic delivery route (i.e., direct diffusion from airway lumen or via recirculation) and variations in ventilatory parameters (i.e., rate, tidal volume, and PEEP) are compared in order to evaluate the importance of interdependence effects. Primary endpoints are measured of respiratory impedance, as well as alterations in the dimensions of specifically identified airway segments from CT images.

In an example embodiment, images are stored on a 4.5 TB server with regular backup. Lung image analysis can include segmentation (semi-automatically separating lung from non-lung structures), analysis of lung density distribution (including quantification of tissue and air volumes), airway extraction and quantification, and image registration mechanics analysis. Airway analysis can be done using programs from VIDA Diagnostics (Iowa City, Iowa, http://www.vidadiagnostics.com). Signal processing for lung impedance analysis and model fitting may be done using customized routines implemented in Matlab.

In some embodiments, it may be difficult to prevent mixing and maintain separation of airway and alveolar gas, and further that any gas destined for the alveolar compartment will pass through the airways. A radiodense Xe gas and CT imaging, or other imaging systems, can be used to visualize the distribution of injected gas in vivo and alveolar plateau (end-tidal) and blood anesthetic concentration can be measured to construct dose-response curves that may distinguish the two effects. Additionally, observing escalating complexity and specificity can insure separation of airways and systemic effects.

A computer-actuated valve system is developed for the precise timing of inhaled anesthetic delivery at variable time intervals in the inspiratory cycle. A key component of this system is a calibrated military drawover vaporizer (Datex-Ohmeda). This vaporizer is compatible with all of the agents used to obtain measurements. Using a mechanical lung model consisting of −180 cc of corrugated tubing ‘deadspace’ in series with two spring-loaded bellows representing the ‘alveolar’ compartment (Ingmar Medical), the ability to control the timing, peak concentration, and duration of inhaled isoflurane delivery for various ranges of conventional ventilator rates and tidal volumes typical of ICU patients can be modeled. Furthermore, any residual concentration of the inhaled anesthetic can be entirely eliminated with exhalation, indicating that re-breathing does not occur within the ventilator circuit. The solenoid valves can be programmed to deliver isoflurane during the last 180 cc of the delivered inspired volume. Moreover, none of the inhaled isoflurane delivered during the last portion of the inspiratory period reached the alveolar compartment. The measurements illustrate delivering inhaled anesthetics in vivo for specified durations in the inspiratory cycle.

Much of the CT imaging work in determining the effects of inhaled anesthetics and other drugs on airway caliber can been done in axial airway slices, and is subject to registration error on repeat measurements as well as problems with oblique cuts. In an example embodiment, rapid high-resolution volumetric scanning and software from VIDA Diagnostics enables quantitative analysis of the airway tree in 3-dimensions. VIDA can identify specific airway pathways from points identified in the periphery back to the trachea and then “straighten” them out to obtain scaled diameters and wall thicknesses as functions of distance along the pathway. A branch numbering scheme allows identification of the same airway in repeat scans. If CT technique is chosen to maximize resolution, airways down to 2-3 mm (4th generation) can be reliably identified and measured.

Image registration is the generation of a mathematical transformation mapping each point on one image set to another. When this is done for the same lung at two volumes, for example end-expiration to end-inspiration, then the Jacobian, calculated from the matrix of partial derivatives of this transform, provides a direct measure of the expansion or contraction of every mapped volume unit. Measurements show that from 5-10 cm H₂O airway pressure, the entire lung expands, although slightly more in the dependent regions. At 15-20 cm H₂O, however, the non-dependent regions are stiffer and expand little while the compliant dependent regions continue to expand. After injury the pattern of expansion is heterogeneous and patchy. The dependent regions are flooded and do not expand from 5-10 cm H₂O; most expansion is in the mid lung. At higher pressure, however, these dependent regions recruit and then can expand. These quantitative measurements are available in 3-D for the entire lung on a voxel level. In one embodiment, peripheral lung regions showing altered expansion are identified first, then VIDA is used to identify the airways feeding that region and their dimensions as well as surrounding tissue stiffness is measured.

In some embodiments, the normal ventilator flow waveform does not contain enough energy at frequencies between 0.1 and 10 Hz to adequately characterize lung input impedance Z, calculated from the Fourier Transform of the instantaneous pressure and flow waveforms measured at the airway opening. By combining specific frequencies such that there is physiologic tidal excursion, in an example embodiment, the EVW is able to maintain gas exchange during conventional ventilation while providing continuous assessment of Z. The EVW differs from other oscillatory waveforms in that it has been modified to allow passive expiration, so Z is calculated from the inspiratory portion of the breath only.

The mechanical impedance spectrum of the lungs or total respiratory system can be very sensitive indicators of bronchoconstriction and response to therapy. Although mathematically Z is a complex number with real and imaginary parts, it can be expressed in terms of effective frequency dependent resistance and elastance. Measurements can be obtained before and 5 minutes following a deep inspiration (DI) to 35 cm H₂O, as well as after administration of intravenous atropine. Under pre and post-DI conditions, both respiratory resistance (Rᵣₛ) and elastance (Eᵣₛ) increased at each frequency with increases in MCh dose. At the lowest frequencies, Rᵣₛ increased ˜50% during the highest dose of MCh compared with baseline due to increases in both airway and tissue resistances. The Eᵣₛ increased by ˜65% as the parenchyma stiffens due to airway-tissue interdependence. In one embodiment, there were no significant differences between the pre and post-DI values of either Rᵣₛ or Eᵣₛ at any MCh dose or frequency. Following administration of intravenous atropine, both Rᵣₛ and Eᵣₛ decrease compared with their respective values at the highest MCh dose of 200 μg/min, and they demonstrate no significant difference compared with their baseline values at any frequency. Thus, the impedance spectra reveal subtleties of airway mechanics and response to therapy not evident from simple Rᵣₛ and Eᵣₛ estimates measured at a single breathing frequency. Coupling these types of data to distributed models of airway and tissue mechanics then allows estimates of the heterogeneity of airway or tissue changes in addition to the overall lung properties.

The methods, systems and media presented herein can be carried out, e.g., via one or more programmable processing units having associated therewith executable instructions held on one or more computer readable medium, RAM, ROM, hard drive, and/or hardware for solving for, deriving and/or applying ranking functions according to the algorithms taught herein. In exemplary embodiments, the hardware, firmware and/or executable code may be provided, e.g., as upgrade graphical module(s) for use in conjunction with existing infrastructure (e.g., existing devices/processing units). Hardware may, e.g., include components and/or logic circuitry for executing the embodiments taught herein as a computing process.

Displays and/or other feedback devices can be included to convey detected/processed data. Thus, in exemplary embodiments, measurements for pressure, flow, volume, percent of anesthetic agent, carbon dioxide partial pressure and oxygen partial pressure, and the like, may be displayed, for example, on a monitor. The display and/or other feedback devices may be stand-alone or may be included as one or more components/graphical modules of the processing unit(s).

The actual software code or control hardware which may be used to implement some of the present embodiments is not intended to limit the scope of such embodiments. For example, certain aspects of the embodiments described herein may be implemented in code using any suitable programming language type using, for example, conventional, graphical (visual), or object-oriented programming techniques. Such code is stored or held on any type of suitable computer-readable medium or media such as, for example, a magnetic or optical storage medium.

As used herein, a “processor,” “processing unit,” “computer” or “computer system” may be, for example, a wireless or wireline variety of a personal computer (PC), microcomputer, minicomputer, server, mainframe, laptop, personal data assistant (PDA), smartphone, tablet, cellular phone, pager, processor, fax machine, scanner, or any other programmable device configured to process data. These devices may also be capable of transmitting and receiving data over a network. Computer systems disclosed herein may include memory for storing certain software applications used in obtaining, processing and communicating data. It can be appreciated that such memory may be internal or external to the disclosed embodiments. The memory may also include storage medium for storing software, including a hard disk, an optical disk, floppy disk, ROM (read only memory), RAM (random access memory), PROM (programmable ROM), EEPROM (electrically erasable PROM), and the like.

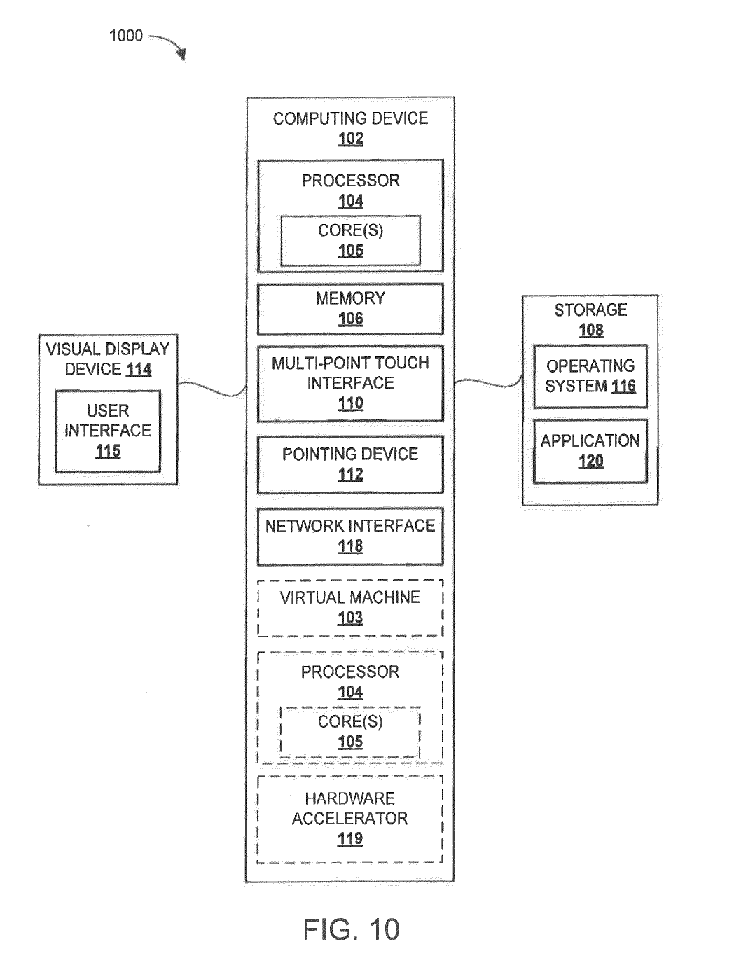

Referring now to FIG. 10, an exemplary computing environment 1000 suitable for practicing exemplary embodiments is depicted. The environment may include a computing device 102 which includes one or more media for storing one or more computer-executable instructions or code for implementing exemplary embodiments. For example, memory 106 included in the computing device 102 may store computer-executable instructions or software, e.g. instructions for implementing and processing every graphical module of the application 120.

The computing device 102 also includes processor 104, and, one or more processor(s) for executing software stored in the memory 106, and other programs for controlling system hardware. Processor 104 or processor(s) each can be a single core processor or multiple core (105) processor. Virtualization can be employed in computing device 102 so that infrastructure and resources in the computing device can be shared dynamically. Virtualized processors may also be used with application 120 and other software in storage 108. A virtual machine 103 can be provided to handle a process running on multiple processors so that the process appears to be using only one computing resource rather than multiple processors. Multiple virtual machines can also be used with one processor. Other computing resources, such as field-programmable gate arrays (FPGA), application specific integrated circuit (ASIC), digital signal processor (DSP), Graphics Processing Unit (GPU), and general-purpose processor (GPP), may also be used for executing code and/or software. A hardware accelerator 119, such as implemented in an ASIC, FPGA, or the like, can additionally be used to speed up the general processing rate of the computing device 102.

The memory 106 may comprise a computer system memory or random access memory, such as DRAM, SRAM, EDO RAM, etc. The memory 106 may comprise other types of memory as well, or combinations thereof. A user may interact with the computing device 102 through a visual display device 114, such as a computer monitor, which may display one or more user interfaces 115. The visual display device 114 may also display other aspects or elements of exemplary embodiments. The computing device 102 may include other I/O devices such a keyboard or a multi-point touch interface 110 and a pointing device 112, for example a mouse, for receiving input from a user. The keyboard 110 and the pointing device 112 may be connected to the visual display device 114. The computing device 102 may include other suitable conventional I/O peripherals. The computing device 102 may further comprise a storage device 108, such as a hard-drive, CD-ROM, or other storage medium for storing an operating system 116 and other programs, e.g., a program 120 including computer executable instructions for delivering an agent at a particular time under specific conditions and for detecting breath phases as described herein.

The computing device 102 may include a network interface 118 to interface to a Local Area Network (LAN), Wide Area Network (WAN) or the Internet through a variety of connections including, but not limited to, standard telephone lines, LAN or WAN links, broadband connections, wireless connections, controller area network (CAN), or some combination of any or all of the above. The network interface 118 may comprise a built-in network adapter, network interface card, PCMCIA network card, card bus network adapter, wireless network adapter, USB network adapter, modem or any other device suitable for interfacing the computing device 102 to any type of network capable of communication and performing the operations described herein. Moreover, the computing device 102 may be any computer system such as a workstation, desktop computer, server, laptop, handheld computer or other form of computing or telecommunications device that is capable of communication and that has sufficient processor power and memory capacity to perform the operations described herein.

The computing device 102 can be running any operating system such as any of the versions of the Microsoft® Windows® operating systems, the different releases of the Unix and Linux operating systems, any version of the MacOS® for Macintosh computers, any version of iOS for iPhones®, any version of Android® OS, Windows Phone OS, any embedded operating system, any real-time operating system, any open source operating system, any proprietary operating system, any operating systems for mobile computing devices, or any other operating system capable of running on the computing device and performing the operations described herein. The operating system may be running in native mode or emulated mode.

The claims should not be read as limited to the described order or elements unless stated to that effect. All embodiments that come within the scope and spirit of the following claims and equivalents thereof are claimed as the invention.

1. A method for targeted delivery of agent for treatment of a respiratory condition, the method comprising:
measuring a respiratory characteristic; and
delivering an agent based on the respiratory characteristic during a respiratory cycle.

2. The method of claim 1, wherein measuring the respiratory characteristic comprises determining a breath phase.

3. The method of claim 2, wherein determining the breath phase comprises:
measuring an airway flow;
comparing the airway flow to a flow increase threshold and a flow decrease threshold;
determining a previous breath phase;
determining the breath phase being an inspiration in response to the airway flow being greater than the flow increase threshold and the previous breath phase being an expiration; and
determining the breath phase being an expiration in response to the airway flow being greater than the flow decrease threshold and the previous breath phase being an inspiration.

4. The method of claim 1, wherein measuring the respiratory characteristic comprises measuring at least one of an airway pressure, an airway flow, a percent of anesthetic agent present, a carbon dioxide partial pressure, and an oxygen partial pressure.

5. The method of claim 1, wherein measuring the respiratory characteristic is triggered by a ventilator cycle.

6. The method of claim 1, further comprising determining a lung resistance, a lung elastance, and an anatomic dead space of the airways based on the respiratory characteristic.

7. The method of claim 1, wherein the agent is an anesthetic.

8. The method of claim 1, wherein the treatment is for the respiratory condition including asthma.

9. The method of claim 1, wherein the treatment is for the respiratory condition including status asthmaticus.

10. The method of claim 1 further comprising connecting a flow delivery system to a ventilator.

11. The method of claim 10 wherein the connecting step includes connecting a vaporizer along a first flow path to the ventilator and connecting a bypass flow path to the ventilator.

12. The method of claim 11 further comprising controlling fluid flow along the first flow path and the bypass flow path with a valve control system.

13. The method of claim 12 wherein the valve control system comprises a driver circuit connected to a first valve and a second valve, the first valve controlling flow between the ventilator and the vaporizer, and the second valve controlling flow between the ventilator and the bypass flow path.

14. The method of claim 12 wherein the valve control system further comprises a computer programmed to deliver valve control signals to a valve control circuit.

15. The method of claim 1 further comprising controlling delivery of a medication into the fluid flow from a ventilator to a patient.

16. The method of claim 13 wherein the valve control system is mounted on a cart.

17. The method of claim 1 further comprising detecting an expiration flow from a respiratory system of a patient.

18. The method of claim 17 wherein the detecting step comprises measuring a gas concentration with a gas analyzer.

19. The method of claim 17 further comprising measuring a level of medication in an expired gas flow, recording measured data in a memory, revising flow parameters with a computer in response to measured data and adjusting valve control system operation with the computer.

20. A system for targeted delivery of agent for treatment of a respiratory condition, the system comprising:
a delivery module configured to:
measure a respiratory characteristic; and
deliver an agent based on the respiratory characteristic during a respiratory cycle.

21. The system of claim 20, further comprising a breath detector module configured to:
measure an airway flow;
compare the airway flow to a flow increase threshold and a flow decrease threshold;
determine a previous breath phase;
determine the breath phase being an inspiration in response to the airway flow being greater than the flow increase threshold and the previous breath phase being an expiration; and
determine the breath phase being an expiration in response to the airway flow being greater than the flow decrease threshold and the previous breath phase being an inspiration.

22. The system of claim 20, wherein the respiratory characteristic comprises at least one of an airway pressure, an airway flow, a percent of anesthetic agent present, a carbon dioxide partial pressure, and an oxygen partial pressure.

23. The system of claim 20 wherein the delivery module further comprises a valve control system connected to a ventilator.

24. The system of claim 20 further comprising a medication delivery device connected to a ventilator.

25. The system of claim 20 wherein the medication delivery device comprises a vaporizer.

26. The system of claim 23 wherein the valve control system comprises a driver circuit connected to a computer, the valve control system operative to control a first valve between the ventilator and a vaporizer, and a bypass valve on a flow path between the ventilator and a patient without the vaporizer.

27. A non-transitory computer-readable storage medium configured to store instructions for targeted delivery of agent for treatment of a respiratory condition, the instructions executable by a processing device, wherein execution of the instructions causes the processing device to implement a method comprising:
measuring a respiratory characteristic; and
delivering an agent based on the respiratory characteristic during a respiratory cycle.

28. The non-transitory computer-readable storage medium of claim 27 further configured to store instructions comprising:
measuring an airway flow;
comparing the airway flow to a flow increase threshold and a flow decrease threshold;
determining a previous breath phase;
determining the breath phase being an inspiration in response to the airway flow being greater than the flow increase threshold and the previous breath phase being an expiration; and
determining the breath phase being an expiration in response to the airway flow being greater than the flow decrease threshold and the previous breath phase being an inspiration.
